(19) United States
(12) Patent Application Publication
(10) Pub. No.: US 2007/0065814 A1
(43) Pub. Date: Mar. 22, 2007

(54) Detecting foot-and-mouth disease virus

(76) Inventor: Eric K. Engelhard, Davis, CA (US)

Correspondence Address: FISH & RICHARDSON P.C., PO BOX 1022, MINNEAPOLIS, MN, 55440-1022, US

(21) Appl. No.: 11/233,245

(22) Filed: Sep. 21, 2005

(51) Int. Cl. C12Q 1/70; C12Q 1/68; C12M 1/34

(52) U.S. Cl. 435/5; 435/6; 435/287.2

(57) ABSTRACT

This document relates to methods and materials involved in determining whether or not an animal contains a foot-and-mouth disease virus. For example, nucleic acid primer pairs, combinations of nucleic acid primer pairs, nucleic acid arrays (e.g., diagnostic cards) containing nucleic acid primer pairs or combinations of nucleic acid primer pairs, methods for making such nucleic acid arrays, and methods for diagnosing animals infected with a foot-and-mouth disease virus are provided.

### BACKGROUND

1. Technical Field

This document relates to methods and materials involved in detecting foot-and-mouth disease virus in cloven-hooved animals (e.g., cattle).

2. Background Information

Cloven-hooved animals such as cattle can become infected with foot-and-mouth disease (FMD) viruses. In some cases, infected animals can become severely ill and even die because of an FMD virus infection. Typically, infected animals experience a lack of weight gain, reduced milk yield, and general un-thriftiness. Properly diagnosing infected animals can help to identify animals for treatment or vaccination and can help to control an outbreak of FMD within, for example, a herd, region, or zoo.

### SUMMARY

This document relates to methods and materials involved in detecting FMD viruses in cloven-hooved animals such as cattle, pigs, sheep, goats, buffalo, deer, and elephants. For example, this document provides nucleic acid primer pairs that can be used in an amplification reaction to detect the presence or absence of a FMD virus' nucleic acid within a sample obtained from the animal being tested. This document also provides combinations of nucleic acid primer pairs, nucleic acid arrays (e.g., diagnostic cards) containing nucleic acid primer pairs or combinations of nucleic acid primer pairs, methods for making such nucleic acid arrays, and methods for diagnosing animals infected with a FMD virus. Such methods and materials can allow, for example, cattle farmers to diagnose an animal as having an FMD virus infection. For example, the nucleic acid primer pairs provided herein can be used to diagnose a cow as having an FMD virus or as being free of FMD viruses. Once diagnosed as having an FMD virus infection, a veterinarian can identify proper treatments or procedures for the infected animal.

The description provided herein is based, in part, on the discovery of nucleic acid primer pairs having the ability to not only amplify particular nucleic acid sequences from FMD viruses, but also to not amplify nucleic acid sequences from non-FMD virus sources such as the host's genome. The description provided herein also is based, in part, on the discovery of sets of nucleic acid primer pairs that can be used simultaneously under the same amplification reaction conditions to amplify different target nucleic acids if present in the sample being tested. For example, a single diagnostic card having ten separate microfluidic chambers, each of which contains a different primer pair provided herein, can be used in a single amplification reaction to detect the presence or absence of up to ten different strains of FMD viruses. Having the ability to test for the presence or absence of multiple strains of FMD viruses using a single diagnostic card and a single amplification reaction can allow, for example, veterinarians to diagnose an animal's condition rapidly in a cost effective manner.

In general, one aspect of this document features a composition comprising, or consisting essentially of, a mixture, wherein the mixture comprises at least one (e.g., 1, 2, 3, 4, 5, 6, 7, 8, 9, 10, 20, 30, 40, 50, 60, 70, 80, 90, 100, 110, 120, 130, 140, 150, or more) primer pair selected from the group consisting of primer pair numbers 1-188 and 189 (e.g., 1, 2, 3, 4, 5, 6, 7, 8, 9, and 10), wherein the primer pair is capable of amplifying a sequence present in a foot-and-mouth disease virus. The mixture can be a solid. The mixture can be a liquid.

In another aspect, this document features an article of manufacture comprising, or consisting essentially of: (a) a substrate defining a microfluidic chamber and (b) a mixture comprising at least one (e.g., 1, 2, 3, 4, 5, 6, 7, 8, 9, 10, 20, 30, 40, 50, 60, 70, 80, 90, 100, 110, 120, 130, 140, 150, or more) primer pair selected from the group consisting of primer pair numbers 1-188 and 189 (e.g., 1, 2, 3, 4, 5, 6, 7, 8, 9, and 10); wherein the mixture is within the chamber; wherein the primer pair is capable of amplifying, within the chamber, a sequence present in a foot-and-mouth disease virus. The mixture can be a solid. The mixture can be a liquid.

In another aspect, this document features a diagnostic card for determining whether or not a cow contains a foot-and-mouth disease virus. The card comprises, or consists essentially of, a plurality of microfluidic chambers, wherein at least one of the microfluidic chambers comprises at least one (e.g., 1, 2, 3, 4, 5, 6, 7, 8, 9, 10, 20, 30, 40, 50, 60, 70, 80, 90, 100, 110, 120, 130, 140, 150, or more) primer pair selected from the group consisting of primer pair numbers 1-188 and 189 (e.g., 1, 2, 3, 4, 5, 6, 7, 8, 9, and 10), which are capable of amplifying, within the chamber, a sequence present in a foot-and-mouth disease virus.

In another aspect, this document features a method for determining whether or not a cloven-hooved animal contains a foot-and-mouth disease virus. The method comprises, or consists essentially of, performing an amplification reaction with at least one (e.g., 1, 2, 3, 4, 5, 6, 7, 8, 9, 10, 20, 30, 40, 50, 60, 70, 80, 90, 100, 110, 120, 130, 140, 150, or more) primer pair selected from the group consisting of primer pair numbers 1-188 and 189 (e.g., 1, 2, 3, 4, 5, 6, 7, 8, 9, and 10) to determine whether or not a sample from the animal contains nucleic acid capable of being amplified with the primer pair, wherein the presence of the nucleic acid indicates that the animal contains a foot-and-mouth disease virus. The animal can be a cow. The sample can be a blood sample.

In another aspect, this document features a method for making an article of manufacture for determining whether or not a cloven-hooved animal contains a foot-and-mouth disease virus. The method comprises, or consists essentially of, (a) providing a substrate defining a microfluidic chamber, and (b) placing a mixture into the chamber to form the article of manufacture, wherein the mixture comprises at least one (e.g., 1, 2, 3, 4, 5, 6, 7, 8, 9, 10, 20, 30, 40, 50, 60, 70, 80, 90, 100, 110, 120, 130, 140, 150, or more) primer pair selected from the group consisting of primer pair numbers 1-188 and 189 (e.g., 1, 2, 3, 4, 5, 6, 7, 8, 9, and 10); wherein the mixture is within the chamber; wherein the primer pair is capable of amplifying, within the chamber, a sequence present in a foot-and-mouth disease virus. The mixture can be a solid. The mixture can be a liquid. The animal can be a cow.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

This document relates to methods and materials involved in detecting FMD viruses in cloven-hooved animals such as cattle, pigs, sheep, goats, buffalo, deer, and elephants. For example, this document provides nucleic acid primer pairs that can be used in an amplification reaction to detect the presence or absence of an FMD virus' nucleic acid within a sample obtained from the animal being tested. This document also provides combinations of nucleic acid primer pairs, nucleic acid arrays (e.g., diagnostic cards) containing nucleic acid primer pairs or combinations of nucleic acid primer pairs, methods for making such nucleic acid arrays, and methods for diagnosing animals infected with an FMD virus.

Nucleic acid primer pairs provided herein are set forth in Table 1. Each primer pair can be used to amplify nucleic acid present in an FMD virus. For example, primer pair number 1 can be used to amplify nucleic acid present in an FMD virus, serotype A. Primer pair number 11 can be used to amplify nucleic acid present in an FMD virus, serotype Asia.

The nucleic acid primer pairs provided herein can be used separately or in combinations. Such combinations can contain 2, 3, 4, 5, 10, 15, 20, 25, 30, 35, 40, 45, 50, 55, 60, 70, 80, 90, 100, or more different nucleic acid primer pairs from Table 1. When making a combination, any two or more of the provided nucleic acid primer pairs can be arranged into any combination. For example, the first nucleic acid primer pair listed in Table 1 for each of the seven serotypes can be used to make a collection of seven different nucleic acid primer pairs. Other combinations include, without limitation, a combination of 89 different nucleic acid primer pairs containing the first 89 nucleic acid primer pairs listed in Table 1; a combination of 15 different nucleic acid primer pair containing the first 15 nucleic acid primer pairs listed in Table 1; and a combination of 100 different nucleic acid primer pairs containing the first 100 nucleic acid primer pairs listed in Table 1.

In some cases, the combination can contain nucleic acid primer pairs 1 through 89 listed in Table 1. Such a combination of nucleic acid primer pairs can be used to make a diagnostic card capable of diagnosing FMD virus infections found in cattle. Such diagnostic cards can be used to determine the presence or absence of an FMD virus within a sample. In some cases, such diagnostic cards can be used to identify a particular FMD virus' serotype and/or strain.

Each nucleic acid primer pair of a combination can be isolated from the other nucleic acid primer pairs of the combination. For example, each nucleic acid primer pair of a combination can be housed within a separate well of a plastic microtiter plate or a separate chamber of a microfluidic card. In some cases, each nucleic acid primer pair of a combination or a subset of nucleic acid primer pairs of a combination can be housed together. For example, five nucleic acid primer pairs of a combination of 50 nucleic acid primer pairs can be housed within a single well of a plastic microtiter plate with the remaining 45 nucleic acid primer pairs being housed within separate wells.

Any method can be used to make each nucleic acid primer of a nucleic acid primer pair. For example, chemical synthesis techniques such as those described elsewhere (Beaucage and Caruthers, Tetrahedron Lett., 22:1859-62 (1981)) can be used. In addition, nucleic acid primers can be ordered from commercial vendors such as MWG Biotech, Invitrogen, and Operon.

This description also provides arrays having at least one of the nucleic acid primer pairs provided herein. Such arrays can be any type of array including, without limitation, two-dimensional arrays, arrays in microtiter plates (e.g., plates with 48, 96, 384, or 1536 wells), arrays fabricated as an arrangement of microfluidic channels and chambers (e.g., a microfluidic card). In some cases, the array can be microfluidic cards with 8 loading ports each connected through microcapillaries to 48 reaction chambers. In some cases, an array provided herein can contain at least 10 different nucleic acid primer pairs set forth in Table 1 (e.g., at least 20, at least 30, at least 50, at least 100, or at least 200 different nucleic acid primer pairs set forth in Table 1).

In addition to containing any one or more of the nucleic acid primer pairs set forth in Table 1 in any combination, an array can contain nucleic acid primer pairs not listed in Table 1. For example, an array can contain a nucleic acid primer pair designed to amplify host nucleic acid (e.g., cattle genomic nucleic acid or mRNA). In some cases, at least 25% (e.g., at least 30%, at least 40%, at least 50%, at least 60%, at least 75%, at least 80%, at least 90%, at least 95%, or 100%) of the nucleic acid primer pairs of an array can be listed in Table 1.

The substrate of an array provided herein can be made of any suitable material (e.g., plastic, glass, silicone, or a metal). In addition, any method can be use to make an array. For example, spotted gelatine or photolithographic techniques can be used to make arrays. In some cases, an array provided herein can be made as follows. A 384-well master plate containing 125 μL of one or more primer pairs in dioinized water at a working concentration of 100 nmole/1 μL of each primer can be constructed. The master plate can be used as a template source, and 1 μL of each master plate well can be transferred to corresponding wells on a 384-well microfluidic card. Spotted reagents can be allowed to dry at room temperature before the final plastic laminate layer of the microfluidic card is attached.

As described herein, the nucleic acid primer pairs set forth in Table 1 can be used to determine whether or not a mammal (e.g., a cow) contains an FMD virus or a set of FMD viruses. For example, a sample can be obtained from a cow and used in an amplification reaction to determine whether or not an FMD virus' nucleic acid is present in the sample. The presence of an amplification product following an amplification reaction using an animal's blood sample and a nucleic acid primer pair designed for an FMD virus can indicate that that sample contains an FMD virus. In such a case, the animal can be diagnosed as being infected with an FMD virus. Any type of sample can be used including, without limitation, a biopsy (e.g., punch biopsy, aspiration biopsy, excision biopsy, needle biopsy, or shave biopsy), a tissue section, lymph fluid, blood, serum, saliva, anal swabs, and synovial fluid samples.

Some sample types can be pre-processed to enhance sample quality, such as the concentration of white blood cells through differential centrifugation. Samples can be processed to concentrate the nucleic acid and render it in a form to facilitate successful PCR reactions. This includes, but is not limited to, common methods to disrupt bilipid membranes, such as the use of detergents, digestion of protein complexes, such as the use of proteinase K, and reduction of polymerase inhibitors through the use of selective affinity columns. Commercial kits for purification of DNA, RNA, or total nucleic acid are readily available from, for example, Qiagen and Roche.

Any type of amplification reaction can be used in conjunction with the nucleic acid primer pairs set forth in Table 1 to detect an FMD virus. For example, common PCR reactions designed to amplify nucleic acid from DNA or RNA can be used. Detection of RNA viruses such as FMD viruses can be accomplished by synthesizing cDNA from RNA sequence templates. cDNA synthesis can be accomplished using standard methods using, for example, RNA-dependant DNA polymerases, such as reverse transcriptase. Such reactions can be primed with random oligonucleotide sequences, such as random hexamers and octamers, or by sequence specific oligonucleotide primers, including the same primers used for the PCR reaction. The cDNA synthesis can be performed in a separate reaction vessel from the subsequent PCR reaction (commonly referred to as two-step rtPCR) or in the same reaction vessel as the PCR reaction (commonly referred to as single-step rtPCR).

Purified DNA and cDNA samples can be pooled and added to a PCR master mix containing water, salt buffers, magnesium ions, nucleotide monomers (dATP, dCTP, dGTP and dTTP), native or engineered Thermus aquaticus DNA-dependant DNA polymerase, and an intercalating dye, such as Sybr Green or LC Green. The master mix and sample can then be added to a single loading port of a microfluidic card and dispersed to all the reaction wells using centrifugation. The cards can then be scored to isolate and seal each reaction chamber prior to thermocycling. The cards can be individually thermocycled using commodity block thermocyclers or many cards thermocycled simultaneously using air- or water-based thermocyclers such as the BioOven or the H2OBIT, respectively.

Positive PCR amplification reactions can be detected during thermocycling for quantitative or qualitative analysis (real time PCR) or after completion of thermocycling (qualitative end-point PCR). Signals can be detected through fluorescence-channel emission of substrate bound intercalating dyes using commodity real-time PCR capable PCR platforms or by end-point reads using microplate scanner platforms. Both types of platforms can be used for melting-point analysis for validation of positive signals.

TABLE 1
Primer pairs that can be used to detect FMD viruses.

| | | | | | |
|---|---|---|---|---|---|
| Primer | | | SEQ | | SEQ |
| Pair | FMDV | | ID | | ID |
| No. | Serotype | Forward Primer Sequence | NO: | Reverse Primer Sequence | NO: |

| | | | | | |
|---|---|---|---|---|---|
| 1 | A | CCTACTACTTCTCTGATTTGGAAATTG | 1 | TGTTTGAACCACTCACAGTGTACTT | 2 |
| 2 | A | AACAAGTACACTGTGAGTGGTTCAA | 3 | AATGATCTTCTGTTTGTGTCTGTCTT | 4 |
| 3 | A | AACAAGTACACTGTGAGTGGTTCAA | 3 | CAATGATCTTCTGTTTGTGTCTGTC | 5 |
| 4 | A | ACTATTTCTCTGACTTGGAAGTTGTG | 6 | GTAGTTGAAGGATGAAGGGAGTTGT | 7 |
| 5 | A | TCAGCCACCTACTATTTCTCTGACT | 8 | GTAGTTGAAGGATGAAGGGAGTTGT | 7 |
| 6 | A | TTCAGCCACCTACTATTTCTCTGAC | 9 | GTAGTTGAAGGATGAAGGGAGTTGT | 7 |
| 7 | A | CCACCTACTATTTCTCTGACTTGGA | 10 | TAGTTGAAGGATGAAGGGAGTTGT | 11 |
| 8 | A | GTCACCACCACTGTTGAGAACTAC | 12 | CACCACAATCTCTAGGTCAGAGAAGTA | 13 |
| 9 | A | GTCACCACCACTGTTGAGAACTAC | 12 | TCTCTAGGTCAGAGAAGTAGTACGTGG | 14 |
| 10 | A | GTCACCACCACTGTTGAGAACTAC | 12 | TTCTAGGTCAGAGAAGTAGTACGTGG | 15 |
| 11 | Asia | GTTGAGAACTACGGAGGAGAAACTC | 16 | CAACCTCCAGGTCTGAGAAGTAGTA | 17 |
| 12 | Asia | AACTACGGAGGAGAAACTCAGACAG | 18 | ACCTCCAGGTCTGAGAAGTAGTACG | 19 |
| 13 | Asia | AGAACTACGGAGGAGAAACTCAGAC | 20 | CAACCTCCAGGTCTGAGAAGTAGTA | 17 |
| 14 | Asia | AGTTGAGAACTACGGAGGAGAGACT | 21 | AACCTCCAGGTCCGAGAAGTAGTA | 22 |
| 15 | Asia | CAGTTGAGAACTACGGAGGAGAGAC | 23 | AACCTCCAGGTCCGAGAAGTAGTA | 22 |
| 16 | C | GTACACTGGCACTACGACCTACAC | 24 | AGAATCGGCCTAGGACAATAGAGTT | 25 |
| 17 | C | AGTTTCTGCACTTGACAACACAAC | 26 | AACTCAGTGATTGTTTCTGCTTTAAC | 27 |
| 18 | C | CAGCCACGTACTACTTCTCTGATCT | 28 | TAGTGTAGGTTGTTGTACCAGTGTACG | 29 |
| 19 | C | GTACACAAGGACAGTATTGTGGGAG | 30 | ACTGGTAGTGTAGGTTGTTGTACCAG | 31 |
| 20 | C | CAGCCACGTACTACTTCTCTGATCT | 28 | CTGGTAGTGTAGGTTGTTGTACCAGT | 32 |
| 21 | C | CACCTACTACTTCTCTGACCTGGAG | 33 | CTTCTGAGCTAACACTTGAAGGTCAC | 34 |
| 22 | O | CACCTACTACTTCTCTGACCTGGAG | 33 | AGAGTTCTTTCTGCCTTCTGAGCTA | 35 |
| 23 | O | CACCTACTACTTCTCTGACCTGGAG | 33 | AGTTCTTTCTGCCTTCTGAGCTAAC | 36 |
| 24 | O | CTGTGACCAATGTGAGAGGTGAC | 37 | ACAATCTTTTGTTTGTGTCTAGCTTC | 38 |
| 25 | O | TGTGAGAGGTGACCTACAAGTGTT | 39 | GTCTTCTGTTTGTTTCTGGCTTC | 40 |
| 26 | O | TCATCATGGACAGATTTGTGAAAGT | 41 | GTCTCCCTCGTGTTTTACTGCTATC | 42 |
| 27 | O | ATCATGGACAGATTTGTGAAAGTGA | 43 | CTCCCTCGTGTTTTACTGCTATCTC | 44 |
| 28 | O | AGATTTGTGAAGATTGGAACCACTA | 45 | GAGTACTTGTTCGTCCCGTTGTA | 46 |
| 29 | O | CAACTTCCTGCCTCTTTCAATTT | 47 | CAATGATCTTCTGTTTGTGTCTGTC | 5 |
| 30 | O | ACTTACTACTTCGCTGATTTAGAAGTGG | 48 | CTAGCACCTGGAGATCACCTCTC | 49 |
| 31 | O | CTTACTACTTCGCTGATTTAGAAGTGG | 50 | ACCGTAGTTAAAGGAGGTAGGCA | 51 |
| 32 | O | GAGTTGCAAGTACAGCAGAGTTGAG | 52 | CAAGAGTTGTTTCATAGGTGCCA | 53 |
| 33 | O | AGTTGCAAGTACAGCAGAGTTGAG | 54 | CAAGAGTTGTTTCATAGGTGCCA | 53 |
| 34 | O | CTTTGATAGCAGTAAAAGGAGACGTT | 55 | AAGTCTCAAGTTGGGAGCATTTCT | 56 |
| 35 | O | TCTTTGATAGCAGTAAAAGGAGACG | 57 | AGTCTCAAGTTGGGAGCATTTCT | 58 |
| 36 | O | AACACACGGACGTCTCATTCATA | 59 | ACTTCTAAGTCAGCGAAATAGTAAGTGG | 60 |
| 37 | O | ACTTACTATTTCGCTGACTTAGAAGTGG | 61 | GTACTTGCAGTTCCCGTTGTAAA | 62 |
| 38 | O | TACTGCTACTTACTACTTCGCAGACCT | 63 | CTAACACTTGCAGGTCACCTCTC | 64 |
| 39 | O | ACTGCTACTTACTACTTCGCAGACCTA | 65 | CCGTTGTAAACAGTAGCCAACAC | 66 |
| 40 | O | AGATTTGTGAAAGTAACACCAAAAGAC | 67 | CGTTGTAAACAGTAGCCAAGACAC | 68 |
| 41 | O | TTAGACAGATTTGTGAAAGTAACACCA | 69 | GTTGTAAACAGTAGCCAAGACACG | 70 |
| 42 | O | GTTTACAACGGGAACTGCAAGTAT | 71 | ACAATCTTTTGTTTGTGTCTAGCTTC | 38 |
| 43 | O | CGTCAGAAACCTCTTAAAGTGAAAG | 72 | CTCAGTGACGATCAAGTTCTTTG | 73 |
| 44 | O | ATCTCAATTCCTTCCCAAAAGTC | 74 | TGATGTTTGCTTTCTCAATGTACTC | 75 |
| 45 | O | AAAGTGACACCAAAAGACCAAATTA | 76 | CGTTGTAGACAGTAGCCAAAACAC | 77 |
| 46 | O | ACCGTGTCTTGGCTACTGTTTAC | 78 | CTTTTGTTTGTGTCTAGCTTCGCT | 79 |
| 47 | O | TTACTCGACTTGCCTTGCCTTAC | 80 | CTATCTTCTGTTTGTGCCTGGCT | 81 |
| 48 | O | CGTCAGAAACCTCTTAAAGTGAAAG | 72 | CTCAGTGACTATCAAGTTCTTTGCT | 82 |
| 49 | O | AGAAACCTCTTAAAGTGAAAGCCAG | 83 | CTCAGTGACTATCAAGTTCTTTGCTT | 84 |
| 50 | O | TTAGACTTGCTCAAGACAAAAGAGAA | 85 | TTGTACTTGCAATCACCGTTGTAG | 86 |
| 51 | SAT1 | GTTAGACTTGCTCAAGACAAAAGAGA | 87 | TTGTACTTGCAATCACCGTTGTAG | 86 |
| 52 | SAT1 | GTTAGACTTGCTCAAGACAAAAGAGA | 87 | CTTGTACTTGCAATCACCGTTGTA | 88 |
| 53 | SAT1 | ACAACAAGATGGTGTTAGACTTGCT | 89 | GTACTTGCAATCACCGTTGTAGGT | 90 |
| 54 | SAT1 | CAGGTGTCTTGCAACAACTTACAAT | 91 | AGGTTTTGTTATCGCTGTCTTGTAG | 92 |
| 55 | SAT1 | AGGTGTCTTGCAACAACTTACAATG | 93 | GTAAATCCTGCCGTAGTTAAAAGTG | 94 |
| 56 | SAT1 | ACACAGGTGTCTTGCAACAACTTAC | 95 | AGGTTTTGTTATCGCTGTCTTGTAG | 92 |
| 57 | SAT1 | TGGTGACTGTAAGTACAAACCCACT | 96 | CAGCTTTAACAGGTCGAAATTACA | 97 |
| 58 | SAT1 | CAACATCCTACAATGGTGACTGTAA | 98 | TACACAACTGTTTGACAGGCTTAGTT | 99 |
| 59 | SAT1 | GTCTTCTCCAAAAACAACACCAC | 100 | GTGTGTAAAGCCTGCCATAGTTAAAG | 101 |
| 60 | SAT1 | AGTCGTCTTCTCCAAAAACAACA | 102 | GTGTGTAAAGCCTGCCATAGTTAAAG | 101 |
| 61 | SAT1 | ACTTTCAACTACGGTAGGATCTACACA | 103 | AACTTTAACAGGTCGAAGTTGCAC | 104 |
| 62 | SAT1 | CTTTCAACTACGGTAGGATCTACACAG | 105 | AACTTTAACAGGTCGAAGTTGCAC | 104 |
| 63 | SAT1 | TCCTACAACGGTGACTGCAAGTA | 106 | GTGATCGTAGTGTGTGAGAAGAGGT | 107 |
| 64 | SAT1 | AACTGTCTACAACGGTGAGTGTAAAT | 108 | TCATCCTGTAGTACACGTCAACACT | 109 |
| 65 | SAT2 | AACTGTCTACAACGGTGAGTGTAAAT | 108 | CATCCTGTAGTACACGTCAACACTT | 110 |
| 66 | SAT2 | TGAGAGCTTCCACCTACTACTTCTG | 111 | TGTGTATTTACACTCACCGTTGTAGA | 112 |
| 67 | SAT2 | CACTGTTTACAACGGTGAGTGTAAGTA | 113 | GCATAGTTGTTTCTCTACCCCAATA | 114 |
| 68 | SAT2 | CACTGTTTACAACGGTGAGTGTAAG | 115 | GCATAGTTGTTTCTCTACCCCAATAG | 116 |
| 69 | SAT2 | TAACACAACTGTACAACCAATACGTG | 117 | GCATAGTTGTTTCTCTACCCCAATA | 114 |
| 70 | SAT2 | CCACCTACTATTTTTGTGACTTGGA | 118 | TGGAGTAGTTACACTCACCGTTGTA | 119 |
| 71 | SAT2 | TATTTTTGTGACTTGGAAATTGCAT | 120 | TGGAGTAGTTACACTCACCGTTGTA | 119 |
| 72 | SAT2 | AGCACTTTCAACTACGGTTACGTG | 121 | ACACAGTTGTTTTTCTACGCCAAT | 122 |
| 73 | SAT2 | GTACTACTTTGCTGACCTTGAAATCG | 123 | ACGTAACCGTAGTTGAAAGTGCTG | 124 |
| 74 | SAT2 | GTTTACAACGGTGAATGCAAATAC | 125 | ATCCTGTAGTACACATCAACGCTACT | 126 |
| 75 | SAT2 | GTTTACAACGGTGAATGCAAATAC | 125 | TGTAGTACACATCAACGCTACTGTCA | 127 |
| 76 | SAT2 | CAAAGCAGTTGATGTGTACTACAGG | 128 | CAACTTTAACAGGTCGTAGTTGCAC | 129 |
| 77 | SAT2 | ACATTCAACTTTGGTCACGTTACTG | 130 | CAACTTTAACAGGTCGTAGTTGCAC | 129 |
| 78 | SAT2 | CCAGCACTTTCAACTACGGTTAC | 131 | ACACAGTTGTTTTTCTACGCCAAT | 122 |
| 79 | SAT2 | GCACTTTCAACTACGGTTACGTG | 132 | ACACAGTTGTTTTTCTACGCCAAT | 122 |
| 80 | SAT2 | GTTTACAACGGTGAATGCAAATAC | 125 | CAACTTTAACAGGTCGAAGTTACACA | 133 |
| 81 | SAT2 | CTATCAACCAGATACAACGGTGAGT | 134 | AGGAGAGGTCTTGGACAGTAGAGTTC | 135 |
| 82 | SAT2 | CTGTCTACTGTCTACAATGGCGAGT | 136 | CTTCATCCGGTAGTAAACATCGACT | 137 |
| 83 | SAT2 | AGGCTGCTGTCTACTGTCTACAATG | 138 | CTTCATCCGGTAGTAAACATCGACT | 137 |
| 84 | SAT2 | AGGCTGCTGTCTACTGTCTACAATG | 138 | ACAGTACAGTTCAGCCCTCTTCAT | 139 |
| 85 | SAT2 | CAGTGTTCTTGCAACAGTCTACAAT | 140 | AATTACACAGTTGTTTATCAGGTGCTAC | 141 |
| 86 | SAT3 | GTCTACAATGGCAACTGCAAATACT | 142 | GTTCTCTTCATCCGGTAGTAGACCT | 143 |
| 87 | SAT3 | GTCTACAATGGCAACTGCAAATACT | 142 | TTCTCTTCATCCGGTAGTAGACCTC | 144 |
| 88 | SAT3 | GTCTACAATGGCAACTGCAAATACT | 142 | ACTTCAACAGGTCGAAATTACACAG | 145 |
| 89 | SAT3 | CTGTAAAGGCTGACACCATCACT | 146 | TCAGGTGCAATGATCTTCTGTTTAC | 147 |
| 90 | Asia | CTGTAAAGGCTGACACCATCACT | 146 | CAGGTGCAATGATCTTCTGTTTAC | 148 |
| 91 | Asia | GTTCTTGACAGGTTTGTGAAACTCA | 149 | GATCAAAAGCTCAGTGATGGTGT | 150 |
| 92 | Asia | GTTCTTGACAGGTTTGTGAAACTCA | 149 | ATCAAAAGCTCAGTGATGGTGTC | 151 |
| 93 | Asia | ACCTCTTTCAACTACGGTGCTGT | 152 | CATAATCTGCTTCTCAGGTGCAA | 153 |
| 94 | Asia | TCTTGACAGGTTTGTGAAACTCACT | 154 | GATCAAAAGCTCAGTGATGGTGT | 150 |
| 95 | Asia | TTTCAACTACGGTGCTGTAAAGG | 155 | CATAATCTGCTTCTCAGGTGCAA | 153 |
| 96 | Asia | GTTCTTGACAGGTTTGTGAAACTC | 156 | GATCAAAAGCTCAGTGATGGTGT | 150 |
| 97 | Asia | GTTCTTGACAGGTTTGTGAAACTC | 156 | ATCAAAAGCTCAGTGATGGTGTC | 151 |
| 98 | Asia | CTTGACAGGTTTGTGAAACTCACT | 157 | GATCAAAAGCTCAGTGATGGTGT | 150 |
| 99 | Asia | CTAGACAACCAGACCAATCCAACT | 158 | CTGAGTAGTGTCAAGAGCTAGCAAAG | 159 |
| 100 | Asia | TAGACAACCAGACCAATCCAACT | 160 | CTGAGTAGTGTCAAGAGCTAGCAAAG | 159 |
| 101 | Asia | CTAGACAACCAGACCAATCCAAC | 161 | CTGAGTAGTGTCAAGAGCTAGCAAAG | 159 |
| 102 | Asia | ACAGTGTACAATGGGAAGACGAC | 162 | CTGAGTAGTGTCAAGAGCTAGCAAAG | 159 |
| 103 | Asia | CTCTAGACAACCAGACCAATCCA | 163 | CTGAGTAGTGTCAAGAGCTAGCAAAG | 159 |
| 104 | Asia | TCTAGACAACCAGACCAATCCAAC | 164 | CTGAGTAGTGTCAAGAGCTAGCAAAG | 159 |
| 105 | Asia | CTCTAGACAACCAGACCAATCCAA | 165 | CTGAGTAGTGTCAAGAGCTAGCAAAG | 159 |
| 106 | Asia | TCTAGACAACCAGACCAATCCAA | 166 | CTGAGTAGTGTCAAGAGCTAGCAAAG | 159 |
| 107 | Asia | GCTCTAGACAACCAGACCAATCC | 167 | CTGAGTAGTGTCAAGAGCTAGCAAAG | 159 |
| 108 | Asia | CTAGACAACCAGACCAATCCAACT | 158 | AGTAGTGTCAAGAGCTAGCAAAGGC | 168 |
| 109 | Asia | ATGCTCTAGACAACCAGACCAATC | 169 | AGTAGTGTCAAGAGCTAGCAAAGGC | 168 |
| 110 | Asia | GATGCTCTAGACAACCAGACCAAT | 170 | AGTAGTGTCAAGAGCTAGCAAAGGC | 168 |
| 111 | Asia | TAGACAACCAGACCAATCCAACT | 160 | AGTAGTGTCAAGAGCTAGCAAAGGC | 168 |
| 112 | Asia | CTAGACAACCAGACCAATCCAAC | 161 | AGTAGTGTCAAGAGCTAGCAAAGGC | 168 |
| 113 | Asia | CTCTAGACAACCAGACCAATCCA | 163 | AGTAGTGTCAAGAGCTAGCAAAGGC | 168 |
| 114 | Asia | TCTAGACAACCAGACCAATCCAAC | 164 | AGTAGTGTCAAGAGCTAGCAAAGGC | 168 |
| 115 | Asia | TGCTCTAGACAACCAGACCAATC | 171 | AGTAGTGTCAAGAGCTAGCAAAGGC | 168 |
| 116 | Asia | GATGCTCTAGACAACCAGACCAA | 172 | AGTAGTGTCAAGAGCTAGCAAAGGC | 168 |
| 117 | Asia | CTCTAGACAACCAGACCAATCCAA | 165 | AGTAGTGTCAAGAGCTAGCAAAGGC | 168 |
| 118 | Asia | ATGCTCTAGACAACCAGACCAAT | 173 | AGTAGTGTCAAGAGCTAGCAAAGGC | 168 |
| 119 | Asia | TCTAGACAACCAGACCAATCCAA | 166 | AGTAGTGTCAAGAGCTAGCAAAGGC | 168 |
| 120 | Asia | AAAGATGCTCTAGACAACCAGACC | 174 | GATCAAAAGCTCAGTAATGGTGTCA | 175 |
| 121 | Asia | CTAGACAACCAAACTAACCCAACTG | 176 | GAGTGGTGTCAAGAGCTAGCAAAG | 177 |
| 122 | Asia | CAAAGATGCTCTAGACAACCAGAC | 178 | GATCAAAAGCTCAGTAATGGTGTCA | 175 |
| 123 | Asia | CCTAGACAACCAAACTAACCCAACT | 179 | GAGTGGTGTCAAGAGCTAGCAAAG | 177 |
| 124 | Asia | ATATGGCTGCCCTTACACTAAAGAC | 180 | GAGTGGTGTCAAGAGCTAGCAAAG | 177 |
| 125 | Asia | GTGTACTGGCGACAGTGTACAAG | 181 | GATCAAAAGCTCAGTAATGGTGTCA | 175 |
| 126 | Asia | ATATGGCTGCCCTTACACTAAAGA | 182 | GAGTGGTGTCAAGAGCTAGCAAAG | 177 |
| 127 | Asia | GATATGGCTGCCCTTACACTAAAG | 183 | GAGTGGTGTCAAGAGCTAGCAAAG | 177 |
| 128 | Asia | GTGTACTGGCGACGGTATACAAC | 184 | GATCAAAAGCTCAGTAATGGTGTCA | 175 |
| 129 | Asia | TATGGCTGCCCTTACACTAAAGACT | 185 | GAGTGGTGTCAAGAGCTAGCAAAG | 177 |
| 130 | Asia | AATCAGACCAATCCAACTGCTTAC | 186 | GAGTGGTGTCAAGAGCTAGCAAAG | 177 |
| 131 | Asia | CTAGACAACCAGACCAATCCAACT | 158 | GAGTGGTGTCAAGAGCTAGCAAAG | 177 |
| 132 | Asia | GACAGTGTACAACGGGAAGACTAC | 187 | GAGTGGTGTCAAGAGCTAGCAAAG | 177 |
| 133 | Asia | TATGGCTGCCCTTACACTAAAGAC | 188 | GAGTGGTGTCAAGAGCTAGCAAAG | 177 |
| 134 | Asia | GATATGGCTGCCCTTACACTAAAGA | 189 | GAGTGGTGTCAAGAGCTAGCAAAG | 177 |
| 135 | Asia | AATCAGACCAATCCAACTGCTTA | 190 | GAGTGGTGTCAAGAGCTAGCAAAG | 177 |
| 136 | Asia | TAGACAACCAGACCAATCCAACT | 160 | GAGTGGTGTCAAGAGCTAGCAAAG | 177 |
| 137 | Asia | CTAGACAACCAGACCAATCCAAC | 161 | GAGTGGTGTCAAGAGCTAGCAAAG | 177 |
| 138 | Asia | TATGGCTGCCCTTACACTAAAGA | 191 | GAGTGGTGTCAAGAGCTAGCAAAG | 177 |
| 139 | Asia | GTGTACTGGCGACAGTGTACAAG | 181 | GAGTGGTGTCAAGAGCTAGCAAAG | 177 |
| 140 | Asia | AGACTACGTACGGGGAAACAACT | 192 | GAGTGGTGTCAAGAGCTAGCAAAG | 177 |
| 141 | Asia | AAGACTACGTACGGGGAAACAACT | 193 | GAGTGGTGTCAAGAGCTAGCAAAG | 177 |
| 142 | Asia | ATCAGACCAATCCAACTGCTTAC | 194 | GAGTGGTGTCAAGAGCTAGCAAAG | 177 |
| 143 | Asia | AGACTACGTACGGGGAAACAACTT | 195 | GAGTGGTGTCAAGAGCTAGCAAAG | 177 |
| 144 | Asia | GATATGGCTGCCCTTACACTAAA | 196 | GAGTGGTGTCAAGAGCTAGCAAAG | 177 |
| 145 | Asia | CTCTAGACAACCAGACCAATCCA | 163 | GAGTGGTGTCAAGAGCTAGCAAAG | 177 |
| 146 | Asia | ACAGTGTACAACGGAAAGACGAC | 197 | GAGTGGTGTCAAGAGCTAGCAAAG | 177 |
| 147 | Asia | TCTAGACAACCAGACCAATCCAAC | 164 | GAGTGGTGTCAAGAGCTAGCAAAG | 177 |
| 148 | Asia | ACTACGTACGGGGAAACAACTTC | 198 | GAGTGGTGTCAAGAGCTAGCAAAG | 177 |
| 149 | Asia | AAGACTACGTACGGGGAAACAAC | 199 | GAGTGGTGTCAAGAGCTAGCAAAG | 177 |
| 150 | Asia | AACAGTGTACAACGGAAAGACGAC | 200 | GAGTGGTGTCAAGAGCTAGCAAAG | 177 |
| 151 | Asia | GACTACGTACGGGGAAACAACTT | 201 | GAGTGGTGTCAAGAGCTAGCAAAG | 177 |
| 152 | Asia | AAAGATGCTCTGGACAACCAAAC | 202 | GATCAAAAGCTCAGTAATGGTGTCA | 175 |
| 153 | Asia | ATGGCTGCCCTTACACTAAAGACT | 203 | GAGTGGTGTCAAGAGCTAGCAAAG | 177 |
| 154 | Asia | ACAGTGTACAACGGGAAGACTACG | 204 | GAGTGGTGTCAAGAGCTAGCAAAG | 177 |
| 155 | Asia | CTCTAGACAACCAGACCAATCCAA | 165 | GAGTGGTGTCAAGAGCTAGCAAAG | 177 |
| 156 | Asia | ATATGGCTGCCCTTACACTAAAGACT | 205 | GAGTGGTGTCAAGAGCTAGCAAAG | 177 |
| 157 | Asia | ATGGCTGCCCTTACACTAAAGAC | 206 | GAGTGGTGTCAAGAGCTAGCAAAG | 177 |
| 158 | Asia | TCTAGACAACCAGACCAATCCAA | 166 | GAGTGGTGTCAAGAGCTAGCAAAG | 177 |
| 159 | Asia | AACAGTGTACAACGGAAAGACGA | 207 | GAGTGGTGTCAAGAGCTAGCAAAG | 177 |
| 160 | Asia | TGATATGGCTGCCCTTACACTAAA | 208 | GAGTGGTGTCAAGAGCTAGCAAAG | 177 |
| 161 | Asia | GCAACAGTGTACAACGGAAAGAC | 209 | GAGTGGTGTCAAGAGCTAGCAAAG | 177 |
| 162 | Asia | CAGTGTACAACGGGAAGACTACG | 210 | GAGTGGTGTCAAGAGCTAGCAAAG | 177 |
| 163 | Asia | ATATGGCTGCCCTTACACTAAAG | 211 | GAGTGGTGTCAAGAGCTAGCAAAG | 177 |
| 164 | Asia | GTGTACAACGGAAAGACGACGTA | 212 | GAGTGGTGTCAAGAGCTAGCAAAG | 177 |
| 165 | Asia | GACAGTGTACAACGGAAAGACGA | 213 | GAGTGGTGTCAAGAGCTAGCAAAG | 177 |
| 166 | Asia | TGGCTGCCCTTACACTAAAGACT | 214 | GAGTGGTGTCAAGAGCTAGCAAAG | 177 |
| 167 | Asia | GAAGACTACGTACGGGGAAACAA | 215 | GAGTGGTGTCAAGAGCTAGCAAAG | 177 |
| 168 | Asia | AAGACGACGTACGGAAAACAAAC | 216 | GAGTGGTGTCAAGAGCTAGCAAAG | 177 |
| 169 | Asia | GACAGTGTACAACGGGAAGACTA | 217 | GAGTGGTGTCAAGAGCTAGCAAAG | 177 |
| 170 | Asia | ATCCAACTGCCTACCAGAAACAG | 218 | GAGTGGTGTCAAGAGCTAGCAAAG | 177 |
| 171 | Asia | ACAGTGTACAAGGGGAAGACGAC | 219 | GAGTGGTGTCAAGAGCTAGCAAAG | 177 |
| 172 | Asia | CAACAGTGTACAACGGAAAGACG | 220 | GAGTGGTGTCAAGAGCTAGCAAAG | 177 |
| 173 | Asia | AAAGATGCTCTAGACAACCAGACC | 174 | ATCAAAAGCTCAGTAATGGTGTCAG | 221 |
| 174 | Asia | ATGCTCTAGACAACCAGACCAAC | 222 | ATCAAAAGCTCAGTAATGGTGTCAG | 221 |
| 175 | Asia | GTGTACTGGCGACAGTGTACAAG | 181 | ATCAAAAGCTCAGTAATGGTGTCAG | 221 |
| 176 | Asia | AGATGCTCTAGACAACCAGACCAA | 223 | ATCAAAAGCTCAGTAATGGTGTCAG | 221 |
| 177 | Asia | GATGCTCTAGACAACCAGACCAA | 172 | ATCAAAAGCTCAGTAATGGTGTCAG | 221 |
| 178 | Asia | GTGTACTGGCGACGGTATACAAC | 184 | ATCAAAAGCTCAGTAATGGTGTCAG | 221 |
| 179 | Asia | CCAAAGATGCTCTAGACAACCAGA | 224 | ATCAAAAGCTCAGTAATGGTGTCAG | 221 |
| 180 | Asia | ACTGACTACCAGAAGCAACCCAT | 225 | ATCAAAAGCTCAGTAATGGTGTCAG | 221 |
| 181 | Asia | CTAGACAACCAAACTAACCCAACTG | 176 | GAGTAGTGTCAAGAGCTAGCAAAGG | 226 |
| 182 | Asia | CTAGACAACCAGACCAATCCAACT | 158 | GAGTAGTGTCAAGAGCTAGCAAAGG | 226 |
| 183 | Asia | ATGCTCTAGACAACCAGACCAATC | 169 | GAGTAGTGTCAAGAGCTAGCAAAGG | 226 |
| 184 | Asia | TAGACAACCAGACCAATCCAACT | 160 | GAGTAGTGTCAAGAGCTAGCAAAGG | 226 |
| 185 | Asia | CTAGACAACCAGACCAATCCAAC | 161 | GAGTAGTGTCAAGAGCTAGCAAAGG | 226 |
| 186 | Asia | CTAGACAACCAGACCAATCCAACT | 158 | TGAGTAGTGTCAAGAGCTAGCAAAG | 227 |
| 187 | Asia | ACAGTGTACAATGGGAAGACGAC | 162 | GAGTAGTGTCAAGAGCTAGCAAAGG | 226 |
| 188 | Asia | TAGACAACCAGACCAATCCAACT | 160 | TGAGTAGTGTCAAGAGCTAGCAAAG | 227 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising a mixture, wherein said mixture comprises at least one primer pair selected from the group consisting of primer pair numbers 1, 2, 3, 4, 5, 6, 7, 8, 9, and 10, wherein said primer pair is capable of amplifying a sequence present in a foot-and-mouth disease virus.

2. The composition of claim 1, wherein said mixture is a solid.

3. The composition of claim 1, wherein said mixture is a liquid.

4. An article of manufacture comprising (a) a substrate defining a microfluidic chamber and (b) a mixture comprising at least one primer pair selected from the group consisting of primer pair numbers 1, 2, 3, 4, 5, 6, 7, 8, 9, and 10; wherein said mixture is within said chamber; wherein said primer pair is capable of amplifying, within said chamber, a sequence present in a foot-and-mouth disease virus.

5. The article of manufacture of claim 4, wherein said mixture is a solid.

6. The article of manufacture of claim 4, wherein said mixture is a liquid.

7. A diagnostic card for determining whether or not a cow contains a foot-and-mouth disease virus, wherein said card comprises a plurality of microfluidic chambers, wherein at least one of said microfluidic chambers comprises at least one primer pair selected from the group consisting of primer pair number 1, 2, 3, 4, 5, 6, 7, 8, 9, and 10, which are capable of amplifying, within said chamber, a sequence present in a foot-and-mouth disease virus.

8. A method for determining whether or not a cloven-hooved animal contains a foot-and-mouth disease virus, wherein said method comprises performing an amplification reaction with at least one primer pair selected from the group consisting of primer pair numbers 1, 2, 3, 4, 5, 6, 7, 8, 9, and 10 to determine whether or not a sample from said animal contains nucleic acid capable of being amplified with said primer pair, wherein the presence of said nucleic acid indicates that said animal contains a foot-and-mouth disease virus.

9. The method of claim 8, wherein said animal is a cow.

10. The method of claim 8, wherein said sample is a blood sample.

11. A method for making an article of manufacture for determining whether or not a cloven-hooved animal contains a foot-and-mouth disease virus, said method comprising:
(a) providing a substrate defining a microfluidic chamber, and
(b) placing a mixture into said chamber to form said article of manufacture, wherein said mixture comprises at least one primer pair selected from the group consisting of primer pair numbers 1, 2, 3, 4, 5, 6, 7, 8, 9, and 10; wherein said mixture is within said chamber; wherein said primer pair is capable of amplifying, within said chamber, a sequence present in a foot-and-mouth disease virus.

12. The method of claim 11, wherein said mixture is a solid.

13. The method of claim 11, wherein said mixture is a liquid.

14. The method of claim 11, wherein said animal is a cow.


SEQUENCE LISTING (227 sequences)

SEQ ID NO: 1 - DNA, 27 bp, Artificial Sequence
  fragment present in foot and mouth virus
cctactacttctctgatttggaaattg

SEQ ID NO: 2 - DNA, 25 bp, Artificial Sequence
  fragment present in foot and mouth virus
tgtttgaaccactcacagtgtactt

SEQ ID NO: 3 - DNA, 25 bp, Artificial Sequence
  fragment present in foot and mouth virus
aacaagtacactgtgagtggttcaa

SEQ ID NO: 4 - DNA, 26 bp, Artificial Sequence
  fragment present in foot and mouth virus
aatgatcttctgtttgtgtctgtctt

SEQ ID NO: 5 - DNA, 25 bp, Artificial Sequence
  fragment present in foot and mouth virus
caatgatcttctgtttgtgtctgtc

SEQ ID NO: 6 - DNA, 26 bp, Artificial Sequence
  fragment present in foot and mouth virus
actatttctctgacttggaagttgtg

SEQ ID NO: 7 - DNA, 25 bp, Artificial Sequence
  fragment present in foot and mouth virus
gtagttgaaggatgaagggagttgt

SEQ ID NO: 8 - DNA, 25 bp, Artificial Sequence
  fragment present in foot and mouth virus
tcagccacctactatttctctgact

SEQ ID NO: 9 - DNA, 25 bp, Artificial Sequence
  fragment present in foot and mouth virus
ttcagccacctactatttctctgac

SEQ ID NO: 10 - DNA, 25 bp, Artificial Sequence
  fragment present in foot and mouth virus
ccacctactatttctctgacttgga

SEQ ID NO: 11 - DNA, 24 bp, Artificial Sequence
  fragment present in foot and mouth virus
tagttgaaggatgaagggagttgt

SEQ ID NO: 12 - DNA, 24 bp, Artificial Sequence
  fragment present in foot and mouth virus
gtcaccaccactgttgagaactac

SEQ ID NO: 13 - DNA, 27 bp, Artificial Sequence
  fragment present in foot and mouth virus
caccacaatctctaggtcagagaagta

SEQ ID NO: 14 - DNA, 27 bp, Artificial Sequence
  fragment present in foot and mouth virus
tctctaggtcagagaagtagtacgtgg

SEQ ID NO: 15 - DNA, 26 bp, Artificial Sequence
  fragment present in foot and mouth virus
ttctaggtcagagaagtagtacgtgg

SEQ ID NO: 16 - DNA, 25 bp, Artificial Sequence
  fragment present in foot and mouth virus
gttgagaactacggaggagaaactc

SEQ ID NO: 17 - DNA, 25 bp, Artificial Sequence
  fragment present in foot and mouth virus
caacctccaggtctgagaagtagta

SEQ ID NO: 18 - DNA, 25 bp, Artificial Sequence
  fragment present in foot and mouth virus
aactacggaggagaaactcagacag

SEQ ID NO: 19 - DNA, 25 bp, Artificial Sequence
  fragment present in foot and mouth virus
acctccaggtctgagaagtagtacg

SEQ ID NO: 20 - DNA, 25 bp, Artificial Sequence
  fragment present in foot and mouth virus
agaactacggaggagaaactcagac

SEQ ID NO: 21 - DNA, 25 bp, Artificial Sequence
  fragment present in foot and mouth virus
agttgagaactacggaggagagact

SEQ ID NO: 22 - DNA, 24 bp, Artificial Sequence
  fragment present in foot and mouth virus
aacctccaggtccgagaagtagta

SEQ ID NO: 23 - DNA, 25 bp, Artificial Sequence
  fragment present in foot and mouth virus
cagttgagaactacggaggagagac

SEQ ID NO: 24 - DNA, 24 bp, Artificial Sequence
  fragment present in foot and mouth virus
gtacactggcactacgacctacac

SEQ ID NO: 25 - DNA, 25 bp, Artificial Sequence
  fragment present in foot and mouth virus
agaatcggcctaggacaatagagtt

SEQ ID NO: 26 - DNA, 24 bp, Artificial Sequence
  fragment present in foot and mouth virus
agtttctgcacttgacaacacaac

SEQ ID NO: 27 - DNA, 26 bp, Artificial Sequence
  fragment present in foot and mouth virus
aactcagtgattgtttctgctttaac

SEQ ID NO: 28 - DNA, 25 bp, Artificial Sequence
  fragment present in foot and mouth virus
cagccacgtactacttctctgatct

SEQ ID NO: 29 - DNA, 27 bp, Artificial Sequence
  fragment present in foot and mouth virus
tagtgtaggttgttgtaccagtgtacg

SEQ ID NO: 30 - DNA, 25 bp, Artificial Sequence
  fragment present in foot and mouth virus
gtacacaaggacagtattgtgggag

SEQ ID NO: 31 - DNA, 26 bp, Artificial Sequence
  fragment present in foot and mouth virus
actggtagtgtaggttgttgtaccag

SEQ ID NO: 32 - DNA, 26 bp, Artificial Sequence
  fragment present in foot and mouth virus
ctggtagtgtaggttgttgtaccagt

SEQ ID NO: 33 - DNA, 25 bp, Artificial Sequence
  fragment present in foot and mouth virus
cacctactacttctctgacctggag

SEQ ID NO: 34 - DNA, 26 bp, Artificial Sequence
  fragment present in foot and mouth virus
cttctgagctaacacttgaaggtcac

SEQ ID NO: 35 - DNA, 25 bp, Artificial Sequence
  fragment present in foot and mouth virus
agagttctttctgccttctgagcta

SEQ ID NO: 36 - DNA, 25 bp, Artificial Sequence
  fragment present in foot and mouth virus
agttctttctgccttctgagctaac

SEQ ID NO: 37 - DNA, 23 bp, Artificial Sequence
  fragment present in foot and mouth virus
ctgtgaccaatgtgagaggtgac

SEQ ID NO: 38 - DNA, 26 bp, Artificial Sequence
  fragment present in foot and mouth virus
acaatcttttgtttgtgtctagcttc

SEQ ID NO: 39 - DNA, 24 bp, Artificial Sequence
  fragment present in foot and mouth virus
tgtgagaggtgacctacaagtgtt

SEQ ID NO: 40 - DNA, 23 bp, Artificial Sequence
  fragment present in foot and mouth virus
gtcttctgtttgtttctggcttc

SEQ ID NO: 41 - DNA, 25 bp, Artificial Sequence
  fragment present in foot and mouth virus
tcatcatggacagatttgtgaaagt

SEQ ID NO: 42 - DNA, 25 bp, Artificial Sequence
  fragment present in foot and mouth virus
gtctccctcgtgttttactgctatc

SEQ ID NO: 43 - DNA, 25 bp, Artificial Sequence
  fragment present in foot and mouth virus
atcatggacagatttgtgaaagtga

SEQ ID NO: 44 - DNA, 25 bp, Artificial Sequence
  fragment present in foot and mouth virus
ctccctcgtgttttactgctatctc

SEQ ID NO: 45 - DNA, 25 bp, Artificial Sequence
  fragment present in foot and mouth virus
agatttgtgaagattggaaccacta

SEQ ID NO: 46 - DNA, 23 bp, Artificial Sequence
  fragment present in foot and mouth virus
gagtacttgttcgtcccgttgta

SEQ ID NO: 47 - DNA, 23 bp, Artificial Sequence
  fragment present in foot and mouth virus
caacttcctgcctctttcaattt

SEQ ID NO: 48 - DNA, 28 bp, Artificial Sequence
  fragment present in foot and mouth virus
acttactacttcgctgatttagaagtgg

SEQ ID NO: 49 - DNA, 23 bp, Artificial Sequence
  fragment present in foot and mouth virus
ctagcacctggagatcacctctc

SEQ ID NO: 50 - DNA, 27 bp, Artificial Sequence
  fragment present in foot and mouth virus
cttactacttcgctgatttagaagtgg

SEQ ID NO: 51 - DNA, 23 bp, Artificial Sequence
  fragment present in foot and mouth virus
accgtagttaaaggaggtaggca

SEQ ID NO: 52 - DNA, 25 bp, Artificial Sequence
  fragment present in foot and mouth virus
gagttgcaagtacagcagagttgag

SEQ ID NO: 53 - DNA, 23 bp, Artificial Sequence
  fragment present in foot and mouth virus
caagagttgtttcataggtgcca

SEQ ID NO: 54 - DNA, 24 bp, Artificial Sequence
  fragment present in foot and mouth virus
agttgcaagtacagcagagttgag

SEQ ID NO: 55 - DNA, 26 bp, Artificial Sequence
  fragment present in foot and mouth virus
ctttgatagcagtaaaaggagacgtt

SEQ ID NO: 56 - DNA, 24 bp, Artificial Sequence
  fragment present in foot and mouth virus
aagtctcaagttgggagcatttct

SEQ ID NO: 57 - DNA, 25 bp, Artificial Sequence
  fragment present in foot and mouth virus
tctttgatagcagtaaaaggagacg

SEQ ID NO: 58 - DNA, 23 bp, Artificial Sequence
  fragment present in foot and mouth virus
agtctcaagttgggagcatttct

SEQ ID NO: 59 - DNA, 23 bp, Artificial Sequence
  fragment present in foot and mouth virus
aacacacggacgtctcattcata

SEQ ID NO: 60 - DNA, 28 bp, Artificial Sequence
  fragment present in foot and mouth virus
acttctaagtcagcgaaatagtaagtgg

SEQ ID NO: 61 - DNA, 28 bp, Artificial Sequence
  fragment present in foot and mouth virus
acttactatttcgctgacttagaagtgg

SEQ ID NO: 62 - DNA, 23 bp, Artificial Sequence
  fragment present in foot and mouth virus
gtacttgcagttcccgttgtaaa

SEQ ID NO: 63 - DNA, 27 bp, Artificial Sequence
  fragment present in foot and mouth virus
tactgctacttactacttcgcagacct

SEQ ID NO: 64 - DNA, 23 bp, Artificial Sequence
  fragment present in foot and mouth virus
ctaacacttgcaggtcacctctc

SEQ ID NO: 65 - DNA, 27 bp, Artificial Sequence
  fragment present in foot and mouth virus
actgctacttactacttcgcagaccta

SEQ ID NO: 66 - DNA, 23 bp, Artificial Sequence
  fragment present in foot and mouth virus
ccgttgtaaacagtagccaacac

SEQ ID NO: 67 - DNA, 27 bp, Artificial Sequence
  fragment present in foot and mouth virus
agatttgtgaaagtaacaccaaaagac

SEQ ID NO: 68 - DNA, 24 bp, Artificial Sequence
  fragment present in foot and mouth virus
cgttgtaaacagtagccaagacac

SEQ ID NO: 69 - DNA, 27 bp, Artificial Sequence
  fragment present in foot and mouth virus
ttagacagatttgtgaaagtaacacca

SEQ ID NO: 70 - DNA, 24 bp, Artificial Sequence
  fragment present in foot and mouth virus
gttgtaaacagtagccaagacacg

SEQ ID NO: 71 - DNA, 24 bp, Artificial Sequence
  fragment present in foot and mouth virus
gtttacaacgggaactgcaagtat

SEQ ID NO: 72 - DNA, 25 bp, Artificial Sequence
  fragment present in foot and mouth virus
cgtcagaaacctcttaaagtgaaag

SEQ ID NO: 73 - DNA, 23 bp, Artificial Sequence
  fragment present in foot and mouth virus
ctcagtgacgatcaagttctttg

SEQ ID NO: 74 - DNA, 23 bp, Artificial Sequence
  fragment present in foot and mouth virus
atctcaattccttcccaaaagtc

SEQ ID NO: 75 - DNA, 25 bp, Artificial Sequence
  fragment present in foot and mouth virus
tgatgtttgctttctcaatgtactc

SEQ ID NO: 76 - DNA, 25 bp, Artificial Sequence
  fragment present in foot and mouth virus
aaagtgacaccaaaagaccaaatta

SEQ ID NO: 77 - DNA, 24 bp, Artificial Sequence
  fragment present in foot and mouth virus
cgttgtagacagtagccaaaacac

SEQ ID NO: 78 - DNA, 23 bp, Artificial Sequence
  fragment present in foot and mouth virus
accgtgtcttggctactgtttac

SEQ ID NO: 79 - DNA, 24 bp, Artificial Sequence
  fragment present in foot and mouth virus
cttttgtttgtgtctagcttcgct

SEQ ID NO: 80 - DNA, 23 bp, Artificial Sequence
  fragment present in foot and mouth virus
ttactcgacttgccttgccttac

SEQ ID NO: 81 - DNA, 23 bp, Artificial Sequence
  fragment present in foot and mouth virus
ctatcttctgtttgtgcctggct

SEQ ID NO: 82 - DNA, 25 bp, Artificial Sequence
  fragment present in foot and mouth virus
ctcagtgactatcaagttctttgct

SEQ ID NO: 83 - DNA, 25 bp, Artificial Sequence
  fragment present in foot and mouth virus
agaaacctcttaaagtgaaagccag

SEQ ID NO: 84 - DNA, 26 bp, Artificial Sequence
  fragment present in foot and mouth virus
ctcagtgactatcaagttctttgctt

SEQ ID NO: 85 - DNA, 26 bp, Artificial Sequence
  fragment present in foot and mouth virus
ttagacttgctcaagacaaaagagaa

SEQ ID NO: 86 - DNA, 24 bp, Artificial Sequence
  fragment present in foot and mouth virus
ttgtacttgcaatcaccgttgtag

SEQ ID NO: 87 - DNA, 26 bp, Artificial Sequence
  fragment present in foot and mouth virus
gttagacttgctcaagacaaaagaga

SEQ ID NO: 88 - DNA, 24 bp, Artificial Sequence
  fragment present in foot and mouth virus
cttgtacttgcaatcaccgttgta

SEQ ID NO: 89 - DNA, 25 bp, Artificial Sequence
  fragment present in foot and mouth virus
acaacaagatggtgttagacttgct

SEQ ID NO: 90 - DNA, 24 bp, Artificial Sequence
  fragment present in foot and mouth virus
gtacttgcaatcaccgttgtaggt

SEQ ID NO: 91 - DNA, 25 bp, Artificial Sequence
  fragment present in foot and mouth virus
caggtgtcttgcaacaacttacaat

SEQ ID NO: 92 - DNA, 25 bp, Artificial Sequence
  fragment present in foot and mouth virus
aggttttgttatcgctgtcttgtag

SEQ ID NO: 93 - DNA, 25 bp, Artificial Sequence
  fragment present in foot and mouth virus
aggtgtcttgcaacaacttacaatg

SEQ ID NO: 94 - DNA, 25 bp, Artificial Sequence
  fragment present in foot and mouth virus
gtaaatcctgccgtagttaaaagtg

SEQ ID NO: 95 - DNA, 25 bp, Artificial Sequence
  fragment present in foot and mouth virus
acacaggtgtcttgcaacaacttac

SEQ ID NO: 96 - DNA, 25 bp, Artificial Sequence
  fragment present in foot and mouth virus
tggtgactgtaagtacaaacccact

SEQ ID NO: 97 - DNA, 24 bp, Artificial Sequence
  fragment present in foot and mouth virus
cagctttaacaggtcgaaattaca

SEQ ID NO: 98 - DNA, 25 bp, Artificial Sequence
  fragment present in foot and mouth virus
caacatcctacaatggtgactgtaa

SEQ ID NO: 99 - DNA, 26 bp, Artificial Sequence
  fragment present in foot and mouth virus
tacacaactgtttgacaggcttagtt

SEQ ID NO: 100 - DNA, 23 bp, Artificial Sequence
  fragment present in foot and mouth virus
gtcttctccaaaaacaacaccac

SEQ ID NO: 101 - DNA, 26 bp, Artificial Sequence
  fragment present in foot and mouth virus
gtgtgtaaagcctgccatagttaaag

SEQ ID NO: 102 - DNA, 23 bp, Artificial Sequence
  fragment present in foot and mouth virus
agtcgtcttctccaaaaacaaca

SEQ ID NO: 103 - DNA, 27 bp, Artificial Sequence
  fragment present in foot and mouth virus
actttcaactacggtaggatctacaca

SEQ ID NO: 104 - DNA, 24 bp, Artificial Sequence
  fragment present in foot and mouth virus
aactttaacaggtcgaagttgcac

SEQ ID NO: 105 - DNA, 27 bp, Artificial Sequence
  fragment present in foot and mouth virus
ctttcaactacggtaggatctacacag

SEQ ID NO: 106 - DNA, 23 bp, Artificial Sequence
  fragment present in foot and mouth virus
tcctacaacggtgactgcaagta

SEQ ID NO: 107 - DNA, 25 bp, Artificial Sequence
  fragment present in foot and mouth virus
gtgatcgtagtgtgtgagaagaggt

SEQ ID NO: 108 - DNA, 26 bp, Artificial Sequence
  fragment present in foot and mouth virus
aactgtctacaacggtgagtgtaaat

SEQ ID NO: 109 - DNA, 25 bp, Artificial Sequence
  fragment present in foot and mouth virus
tcatcctgtagtacacgtcaacact

SEQ ID NO: 110 - DNA, 25 bp, Artificial Sequence
  fragment present in foot and mouth virus
catcctgtagtacacgtcaacactt

SEQ ID NO: 111 - DNA, 25 bp, Artificial Sequence
  fragment present in foot and mouth virus
tgagagcttccacctactacttctg

SEQ ID NO: 112 - DNA, 26 bp, Artificial Sequence
  fragment present in foot and mouth virus
tgtgtatttacactcaccgttgtaga

SEQ ID NO: 113 - DNA, 27 bp, Artificial Sequence
  fragment present in foot and mouth virus
cactgtttacaacggtgagtgtaagta

SEQ ID NO: 114 - DNA, 25 bp, Artificial Sequence
  fragment present in foot and mouth virus
gcatagttgtttctctaccccaata

SEQ ID NO: 115 - DNA, 25 bp, Artificial Sequence
  fragment present in foot and mouth virus
cactgtttacaacggtgagtgtaag

SEQ ID NO: 116 - DNA, 26 bp, Artificial Sequence
  fragment present in foot and mouth virus
gcatagttgtttctctaccccaatag

SEQ ID NO: 117 - DNA, 26 bp, Artificial Sequence
  fragment present in foot and mouth virus
taacacaactgtacaaccaatacgtg

SEQ ID NO: 118 - DNA, 25 bp, Artificial Sequence
  fragment present in foot and mouth virus
ccacctactatttttgtgacttgga

SEQ ID NO: 119 - DNA, 25 bp, Artificial Sequence
  fragment present in foot and mouth virus
tggagtagttacactcaccgttgta

SEQ ID NO: 120 - DNA, 25 bp, Artificial Sequence
  fragment present in foot and mouth virus
tatttttgtgacttggaaattgcat

SEQ ID NO: 121 - DNA, 24 bp, Artificial Sequence
  fragment present in foot and mouth virus
agcactttcaactacggttacgtg

SEQ ID NO: 122 - DNA, 24 bp, Artificial Sequence
  fragment present in foot and mouth virus
acacagttgtttttctacgccaat

SEQ ID NO: 123 - DNA, 26 bp, Artificial Sequence
  fragment present in foot and mouth virus
gtactactttgctgaccttgaaatcg

SEQ ID NO: 124 - DNA, 24 bp, Artificial Sequence
  fragment present in foot and mouth virus
acgtaaccgtagttgaaagtgctg

SEQ ID NO: 125 - DNA, 24 bp, Artificial Sequence
  fragment present in foot and mouth virus
gtttacaacggtgaatgcaaatac

SEQ ID NO: 126 - DNA, 26 bp, Artificial Sequence
  fragment present in foot and mouth virus
atcctgtagtacacatcaacgctact

SEQ ID NO: 127 - DNA, 26 bp, Artificial Sequence
  fragment present in foot and mouth virus
tgtagtacacatcaacgctactgtca

SEQ ID NO: 128 - DNA, 25 bp, Artificial Sequence
  fragment present in foot and mouth virus
caaagcagttgatgtgtactacagg

SEQ ID NO: 129 - DNA, 25 bp, Artificial Sequence
  fragment present in foot and mouth virus
caactttaacaggtcgtagttgcac

SEQ ID NO: 130 - DNA, 25 bp, Artificial Sequence
  fragment present in foot and mouth virus
acattcaactttggtcacgttactg

SEQ ID NO: 131 - DNA, 23 bp, Artificial Sequence
  fragment present in foot and mouth virus
ccagcactttcaactacggttac

SEQ ID NO: 132 - DNA, 23 bp, Artificial Sequence
  fragment present in foot and mouth virus
gcactttcaactacggttacgtg

SEQ ID NO: 133 - DNA, 26 bp, Artificial Sequence
  fragment present in foot and mouth virus
caactttaacaggtcgaagttacaca

SEQ ID NO: 134 - DNA, 25 bp, Artificial Sequence
  fragment present in foot and mouth virus
ctatcaaccagatacaacggtgagt

SEQ ID NO: 135 - DNA, 26 bp, Artificial Sequence
  fragment present in foot and mouth virus
aggagaggtcttggacagtagagttc

SEQ ID NO: 136 - DNA, 25 bp, Artificial Sequence
  fragment present in foot and mouth virus
ctgtctactgtctacaatggcgagt

SEQ ID NO: 137 - DNA, 25 bp, Artificial Sequence
  fragment present in foot and mouth virus
cttcatccggtagtaaacatcgact

SEQ ID NO: 138 - DNA, 25 bp, Artificial Sequence
  fragment present in foot and mouth virus
aggctgctgtctactgtctacaatg

SEQ ID NO: 139 - DNA, 24 bp, Artificial Sequence
  fragment present in foot and mouth virus
acagtacagttcagccctcttcat

SEQ ID NO: 140 - DNA, 25 bp, Artificial Sequence
  fragment present in foot and mouth virus
cagtgttcttgcaacagtctacaat

SEQ ID NO: 141 - DNA, 28 bp, Artificial Sequence
  fragment present in foot and mouth virus
aattacacagttgtttatcaggtgctac

SEQ ID NO: 142 - DNA, 25 bp, Artificial Sequence
  fragment present in foot and mouth virus
gtctacaatggcaactgcaaatact

SEQ ID NO: 143 - DNA, 25 bp, Artificial Sequence
  fragment present in foot and mouth virus
gttctcttcatccggtagtagacct

SEQ ID NO: 144 - DNA, 25 bp, Artificial Sequence
  fragment present in foot and mouth virus
ttctcttcatccggtagtagacctc

SEQ ID NO: 145 - DNA, 25 bp, Artificial Sequence
  fragment present in foot and mouth virus
acttcaacaggtcgaaattacacag

SEQ ID NO: 146 - DNA, 23 bp, Artificial Sequence
  fragment present in foot and mouth virus
ctgtaaaggctgacaccatcact

SEQ ID NO: 147 - DNA, 25 bp, Artificial Sequence
  fragment present in foot and mouth virus
tcaggtgcaatgatcttctgtttac

SEQ ID NO: 148 - DNA, 24 bp, Artificial Sequence
  fragment present in foot and mouth virus
caggtgcaatgatcttctgtttac

SEQ ID NO: 149 - DNA, 25 bp, Artificial Sequence
  fragment present in foot and mouth virus
gttcttgacaggtttgtgaaactca

SEQ ID NO: 150 - DNA, 23 bp, Artificial Sequence
  fragment present in foot and mouth virus
gatcaaaagctcagtgatggtgt

SEQ ID NO: 151 - DNA, 23 bp, Artificial Sequence
  fragment present in foot and mouth virus
atcaaaagctcagtgatggtgtc

SEQ ID NO: 152 - DNA, 23 bp, Artificial Sequence
  fragment present in foot and mouth virus
acctctttcaactacggtgctgt

SEQ ID NO: 153 - DNA, 23 bp, Artificial Sequence
  fragment present in foot and mouth virus
cataatctgcttctcaggtgcaa

SEQ ID NO: 154 - DNA, 25 bp, Artificial Sequence
  fragment present in foot and mouth virus
tcttgacaggtttgtgaaactcact

SEQ ID NO: 155 - DNA, 23 bp, Artificial Sequence
  fragment present in foot and mouth virus
tttcaactacggtgctgtaaagg

SEQ ID NO: 156 - DNA, 24 bp, Artificial Sequence
  fragment present in foot and mouth virus
gttcttgacaggtttgtgaaactc

SEQ ID NO: 157 - DNA, 24 bp, Artificial Sequence
  fragment present in foot and mouth virus
cttgacaggtttgtgaaactcact

SEQ ID NO: 158 - DNA, 24 bp, Artificial Sequence
  fragment present in foot and mouth virus
ctagacaaccagaccaatccaact

SEQ ID NO: 159 - DNA, 26 bp, Artificial Sequence
  fragment present in foot and mouth virus
ctgagtagtgtcaagagctagcaaag

SEQ ID NO: 160 - DNA, 23 bp, Artificial Sequence
  fragment present in foot and mouth virus
tagacaaccagaccaatccaact

SEQ ID NO: 161 - DNA, 23 bp, Artificial Sequence
  fragment present in foot and mouth virus
ctagacaaccagaccaatccaac

SEQ ID NO: 162 - DNA, 23 bp, Artificial Sequence
  fragment present in foot and mouth virus
acagtgtacaatgggaagacgac

SEQ ID NO: 163 - DNA, 23 bp, Artificial Sequence
  fragment present in foot and mouth virus
ctctagacaaccagaccaatcca

SEQ ID NO: 164 - DNA, 24 bp, Artificial Sequence
  fragment present in foot and mouth virus
tctagacaaccagaccaatccaac

SEQ ID NO: 165 - DNA, 24 bp, Artificial Sequence
  fragment present in foot and mouth virus
ctctagacaaccagaccaatccaa

SEQ ID NO: 166 - DNA, 23 bp, Artificial Sequence
  fragment present in foot and mouth virus
tctagacaaccagaccaatccaa

SEQ ID NO: 167 - DNA, 23 bp, Artificial Sequence
  fragment present in foot and mouth virus
gctctagacaaccagaccaatcc

SEQ ID NO: 168 - DNA, 25 bp, Artificial Sequence
  fragment present in foot and mouth virus
agtagtgtcaagagctagcaaaggc

SEQ ID NO: 169 - DNA, 24 bp, Artificial Sequence
  fragment present in foot and mouth virus
atgctctagacaaccagaccaatc

SEQ ID NO: 170 - DNA, 24 bp, Artificial Sequence
  fragment present in foot and mouth virus
gatgctctagacaaccagaccaat

SEQ ID NO: 171 - DNA, 23 bp, Artificial Sequence
  fragment present in foot and mouth virus
tgctctagacaaccagaccaatc

SEQ ID NO: 172 - DNA, 23 bp, Artificial Sequence
  fragment present in foot and mouth virus
gatgctctagacaaccagaccaa

SEQ ID NO: 173 - DNA, 23 bp, Artificial Sequence
  fragment present in foot and mouth virus
atgctctagacaaccagaccaat

SEQ ID NO: 174 - DNA, 24 bp, Artificial Sequence
  fragment present in foot and mouth virus
aaagatgctctagacaaccagacc

SEQ ID NO: 175 - DNA, 25 bp, Artificial Sequence
  fragment present in foot and mouth virus
gatcaaaagctcagtaatggtgtca

SEQ ID NO: 176 - DNA, 25 bp, Artificial Sequence
  fragment present in foot and mouth virus
ctagacaaccaaactaacccaactg

SEQ ID NO: 177 - DNA, 24 bp, Artificial Sequence
  fragment present in foot and mouth virus
gagtggtgtcaagagctagcaaag

SEQ ID NO: 178 - DNA, 24 bp, Artificial Sequence
  fragment present in foot and mouth virus
caaagatgctctagacaaccagac

SEQ ID NO: 179 - DNA, 25 bp, Artificial Sequence
  fragment present in foot and mouth virus
cctagacaaccaaactaacccaact

SEQ ID NO: 180 - DNA, 25 bp, Artificial Sequence
  fragment present in foot and mouth virus
atatggctgcccttacactaaagac

SEQ ID NO: 181 - DNA, 23 bp, Artificial Sequence
  fragment present in foot and mouth virus
gtgtactggcgacagtgtacaag

SEQ ID NO: 182 - DNA, 24 bp, Artificial Sequence
  fragment present in foot and mouth virus
atatggctgcccttacactaaaga

SEQ ID NO: 183 - DNA, 24 bp, Artificial Sequence
  fragment present in foot and mouth virus
gatatggctgcccttacactaaag

SEQ ID NO: 184 - DNA, 23 bp, Artificial Sequence
  fragment present in foot and mouth virus
gtgtactggcgacggtatacaac

SEQ ID NO: 185 - DNA, 25 bp, Artificial Sequence
  fragment present in foot and mouth virus
tatggctgcccttacactaaagact

SEQ ID NO: 186 - DNA, 24 bp, Artificial Sequence
  fragment present in foot and mouth virus
aatcagaccaatccaactgcttac

SEQ ID NO: 187 - DNA, 24 bp, Artificial Sequence
  fragment present in foot and mouth virus
gacagtgtacaacgggaagactac

SEQ ID NO: 188 - DNA, 24 bp, Artificial Sequence
  fragment present in foot and mouth virus
tatggctgcccttacactaaagac

SEQ ID NO: 189 - DNA, 25 bp, Artificial Sequence
  fragment present in foot and mouth virus
gatatggctgcccttacactaaaga

SEQ ID NO: 190 - DNA, 23 bp, Artificial Sequence
  fragment present in foot and mouth virus
aatcagaccaatccaactgctta

SEQ ID NO: 191 - DNA, 23 bp, Artificial Sequence
  fragment present in foot and mouth virus
tatggctgcccttacactaaaga

SEQ ID NO: 192 - DNA, 23 bp, Artificial Sequence
  fragment present in foot and mouth virus
agactacgtacggggaaacaact

SEQ ID NO: 193 - DNA, 24 bp, Artificial Sequence
  fragment present in foot and mouth virus
aagactacgtacggggaaacaact

SEQ ID NO: 194 - DNA, 23 bp, Artificial Sequence
  fragment present in foot and mouth virus
atcagaccaatccaactgcttac

SEQ ID NO: 195 - DNA, 24 bp, Artificial Sequence
  fragment present in foot and mouth virus
agactacgtacggggaaacaactt

SEQ ID NO: 196 - DNA, 23 bp, Artificial Sequence
  fragment present in foot and mouth virus
gatatggctgcccttacactaaa

SEQ ID NO: 197 - DNA, 23 bp, Artificial Sequence
  fragment present in foot and mouth virus
acagtgtacaacggaaagacgac

SEQ ID NO: 198 - DNA, 23 bp, Artificial Sequence
  fragment present in foot and mouth virus
actacgtacggggaaacaacttc

SEQ ID NO: 199 - DNA, 23 bp, Artificial Sequence
  fragment present in foot and mouth virus
aagactacgtacggggaaacaac

SEQ ID NO: 200 - DNA, 24 bp, Artificial Sequence
  fragment present in foot and mouth virus
aacagtgtacaacggaaagacgac

SEQ ID NO: 201 - DNA, 23 bp, Artificial Sequence
  fragment present in foot and mouth virus
gactacgtacggggaaacaactt

SEQ ID NO: 202 - DNA, 23 bp, Artificial Sequence
  fragment present in foot and mouth virus
aaagatgctctggacaaccaaac

SEQ ID NO: 203 - DNA, 24 bp, Artificial Sequence
  fragment present in foot and mouth virus
atggctgcccttacactaaagact

SEQ ID NO: 204 - DNA, 24 bp, Artificial Sequence
  fragment present in foot and mouth virus
acagtgtacaacgggaagactacg

SEQ ID NO: 205 - DNA, 26 bp, Artificial Sequence
  fragment present in foot and mouth virus
atatggctgcccttacactaaagact

SEQ ID NO: 206 - DNA, 23 bp, Artificial Sequence
  fragment present in foot and mouth virus
atggctgcccttacactaaagac

SEQ ID NO: 207 - DNA, 23 bp, Artificial Sequence
  fragment present in foot and mouth virus
aacagtgtacaacggaaagacga

SEQ ID NO: 208 - DNA, 24 bp, Artificial Sequence
  fragment present in foot and mouth virus
tgatatggctgcccttacactaaa

SEQ ID NO: 209 - DNA, 23 bp, Artificial Sequence
  fragment present in foot and mouth virus
gcaacagtgtacaacggaaagac

SEQ ID NO: 210 - DNA, 23 bp, Artificial Sequence
  fragment present in foot and mouth virus
cagtgtacaacgggaagactacg

SEQ ID NO: 211 - DNA, 23 bp, Artificial Sequence
  fragment present in foot and mouth virus
atatggctgcccttacactaaag

SEQ ID NO: 212 - DNA, 23 bp, Artificial Sequence
  fragment present in foot and mouth virus
gtgtacaacggaaagacgacgta

SEQ ID NO: 213 - DNA, 23 bp, Artificial Sequence
  fragment present in foot and mouth virus
gacagtgtacaacggaaagacga

SEQ ID NO: 214 - DNA, 23 bp, Artificial Sequence
  fragment present in foot and mouth virus
tggctgcccttacactaaagact

SEQ ID NO: 215 - DNA, 23 bp, Artificial Sequence
  fragment present in foot and mouth virus
gaagactacgtacggggaaacaa

SEQ ID NO: 216 - DNA, 23 bp, Artificial Sequence
  fragment present in foot and mouth virus
aagacgacgtacggaaaacaaac

SEQ ID NO: 217 - DNA, 23 bp, Artificial Sequence
  fragment present in foot and mouth virus
gacagtgtacaacgggaagacta

SEQ ID NO: 218 - DNA, 23 bp, Artificial Sequence
  fragment present in foot and mouth virus
atccaactgcctaccagaaacag

SEQ ID NO: 219 - DNA, 23 bp, Artificial Sequence
  fragment present in foot and mouth virus
acagtgtacaaggggaagacgac

SEQ ID NO: 220 - DNA, 23 bp, Artificial Sequence
  fragment present in foot and mouth virus
caacagtgtacaacggaaagacg

SEQ ID NO: 221 - DNA, 25 bp, Artificial Sequence
  fragment present in foot and mouth virus
atcaaaagctcagtaatggtgtcag

SEQ ID NO: 222 - DNA, 23 bp, Artificial Sequence
  fragment present in foot and mouth virus
atgctctagacaaccagaccaac

SEQ ID NO: 223 - DNA, 24 bp, Artificial Sequence
  fragment present in foot and mouth virus
agatgctctagacaaccagaccaa

SEQ ID NO: 224 - DNA, 24 bp, Artificial Sequence
  fragment present in foot and mouth virus
ccaaagatgctctagacaaccaga

SEQ ID NO: 225 - DNA, 23 bp, Artificial Sequence
  fragment present in foot and mouth virus
actgactaccagaagcaacccat

SEQ ID NO: 226 - DNA, 25 bp, Artificial Sequence
  fragment present in foot and mouth virus
gagtagtgtcaagagctagcaaagg

SEQ ID NO: 227 - DNA, 25 bp, Artificial Sequence
  fragment present in foot and mouth virus
tgagtagtgtcaagagctagcaaag